(19) United States
(12) Patent Application Publication
(10) Pub. No.: US 2024/0350004 A1
(43) Pub. Date: Oct. 24, 2024

(54) MODULE FOR TRACKING EYEBALL AND EXTENDED REALITY DEVICE

(71) Applicant: Beijing Zitiao Network Technology Co., Ltd., Beijing (CN)

(72) Inventors: Wenlong BAI, Beijing (CN); Jiu XIA, Beijing (CN)

(21) Appl. No.: 18/640,897

(22) Filed: Apr. 19, 2024

(30) Foreign Application Priority Data
Apr. 19, 2023 (CN) 202310423461.9

(51) Int. Cl. A61B 3/113; A61B 90/00

(52) U.S. Cl. CPC A61B 3/113; A61B 90/36; A61B 2090/365

(57) ABSTRACT

The present disclosure provides a module for tracking an eyeball and an extended reality device. Specifically, the module for tracking an eyeball includes luminescent light sources configured to form light spots on an eye of a user; an image acquiring unit configured to acquire a tracking image of a target area; and an analyzing unit configured to determine a pose of the eyeball based on an eye image and a light spot image in the tracking image; wherein, the luminescent light sources comprise a first light source and form a first light spot on the eye, so as to determine a position of a luminescent light source corresponding to at least part of light spots in the light spot image based on the first light spot.

### CROSS REFERENCE

The present application claims priority to Chinese Patent Application No. 202310423461.9 filed on Apr. 19, 2023 and entitled “MODULE FOR TRACKING EYEBALL AND EXTENDED REALITY DEVICE”, the entirety of which is incorporated herein by reference.

### FIELD

The present disclosure relates to a technical field of extended reality, and in particular to a module for tracking an eyeball and an extended reality device.

### BACKGROUND

Extended reality (XR for short) refers to a combination of reality and virtuality through computers to create a virtual environment that allows human-computer interaction, it is also a collective name for technologies such as Augmented Reality (AR for short), Virtual Reality (VR for short) and Mix Reality (MR for short).

With the rapid development of XR devices, the eyeball tracking technology is gradually becoming popular in XR technology. Adding the eye tracking technology to XR devices (especially VR) may achieve high-efficiency gaze point rendering, which greatly improves rendering efficiency, improves resolution, reduces computational resource consumption and save power consumption. At the same time, eye tracking may also introduce fresher and more realistic XR interactive experiences. For this reason, improving the recognition accuracy and recognition rate of eye tracking is an important development direction.

### SUMMARY

In view of this, the purpose of the present disclosure is to propose a module for tracking an eyeball and an extended reality device.

Based on the above purpose, in a first aspect, an embodiment of the present disclosure provides a module for tracking an eyeball, including:
a luminescent light source configured to form a light spot on an eye of a user;
an image acquiring unit configured to acquire a tracking image of a target area; and
an analyzing unit configured to determine a pose of the eyeball based on an eye image and a light spot image in the tracking image;
wherein, the luminescent light source comprises a first light source and forms a first light spot on the eye, so as to determine a position of a luminescent light source corresponding to at least part of light spots in the light spot image based on the first light spot.

In a second aspect, an embodiment of the present disclosure provides an extended reality device, including:
a body; and
a module for tracking an eyeball of as described in the first aspect disposed on the body.

As can be seen from the above, in the module for tracking an eyeball and extended reality device provided by exemplary embodiments of the present disclosure, by adding a first light source and forming a first light spot on the eye, a position of a luminescent light source corresponding to at least part of the light spots in the light spot image are determined based on the first spot, and thus light spot confusion is effectively prevented, thereby improving algorithm recognition and solving efficiency, and achieving beneficial effects of improving the eye tracking accuracy, increasing the available range of eye tracking, simplifying the algorithm, and reducing system power consumption.

### BRIEF DESCRIPTION OF THE DRAWINGS

In order to explain the technical solutions in the present disclosure or the related technologies more clearly, the drawings needed to be used in the description of embodiments or related technologies will be briefly introduced below. Obviously, the drawings in the following description are only the embodiments of the present disclosure, for those of ordinary skill in the art, other drawings may also be obtained based on these drawings without creative efforts.

FIG. 1A illustrates a principle schematic diagram of an eye tracking solution provided by an exemplary embodiment of the present disclosure;

FIG. 1B illustrates a schematic side view of an eye tracking solution provided by an exemplary embodiment of the present disclosure;

FIG. 2A illustrates a schematic structural diagram of a module for tracking an eyeball provided by related technology;

FIG. 2B illustrates a schematic diagram of a tracking image obtained by the module for tracking the eyeball provided by related technologies;

FIG. 3A illustrates a schematic structural diagram of a module for tracking an eyeball provided by an exemplary embodiment of the present disclosure;

FIG. 3B illustrates a schematic diagram of a tracking image obtained by another module for tracking an eyeball provided by an exemplary embodiment of the present disclosure;

FIG. 3C illustrates a schematic structural diagram of a first light source provided by an exemplary embodiment of the present disclosure;

FIG. 3D illustrates a schematic structural diagram of another first light source provided by an exemplary embodiment of the present disclosure;

FIG. 4A illustrates a schematic structural diagram of an extended reality device provided by an exemplary embodiment of the present disclosure;

FIG. 4B illustrates a schematic structural diagram of another extended reality device provided by an exemplary embodiment of the present disclosure;

FIG. 4C illustrates a partial structural schematic diagram of a body of an extended reality device provided by an exemplary embodiment of the present disclosure;

FIG. 4D illustrates a partial structural schematic diagram of a body of another extended reality device provided by an exemplary embodiment of the present disclosure;

FIG. 4E illustrates a schematic cross-sectional view along plane A-A in FIG. 4D.

### DETAILED DESCRIPTION

In order to make the purpose, technical solutions and advantages of the present disclosure clearer, the present disclosure will be further described in detail below in combination with specific embodiments with reference to the accompanying drawings.

It should be noted that, unless otherwise defined, the technical terms or scientific terms used in the embodiments of the present disclosure should have common meanings understood by those with ordinary skills in the art to which this disclosure belongs. “First”, “second” and similar words used in embodiments of this disclosure do not indicate any order, quantity or importance, but are only used to distinguish different components. A Words such as “include” or “comprise” means that the elements or things appearing before the word include the elements or things listed after the word and their equivalents, without excluding other elements or things. Words such as “connected” or “connecting” are not limited to physical or mechanical connections, but may include electrical connections, whether direct or indirect. “Up”, “down”, “left”, “right”, etc. are only used to express relative positional relationships. When the absolute position of the described object changes, the relative positional relationship may also change accordingly.

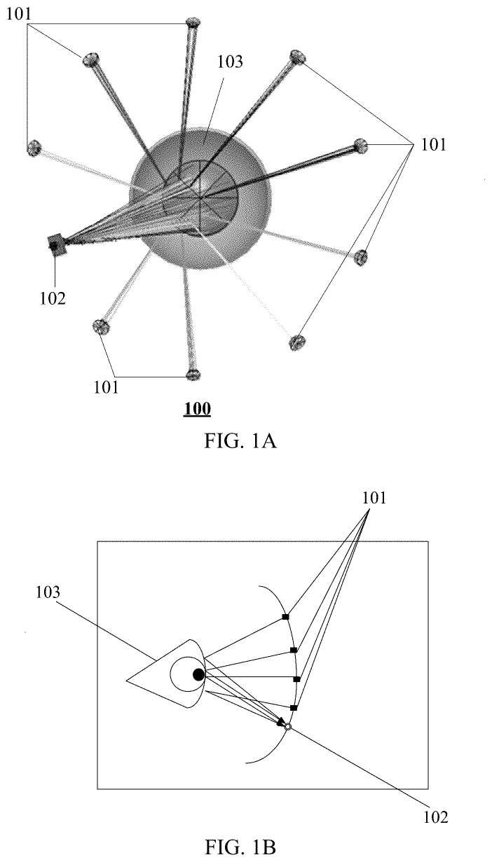

FIG. 1A illustrates a principle schematic diagram of an eye tracking 100 solution provided by exemplary an embodiment of the present disclosure. FIG. 1B illustrates a schematic side view of an eye tracking solution provided by an exemplary embodiment of the present disclosure. By placing a circle of infrared light-emitting diodes 101 (LEDs) in front of an eyeball 103 and surrounding the eyeball, the infrared LEDs 101 emit infrared light and infrared light spots are formed after the infrared light is irradiated onto the eyeball. The light reflected by the eyeball 103 may enter an infrared camera 102 facing the eyeball 103, whereby the infrared camera 102 obtains an image including infrared light spots and the eyeball. Through position and angle relationships between the eyeball and the infrared light spots in the image, the direction and attitude of the eyeball can be calculated using mathematical methods to achieve eyeball tracking.

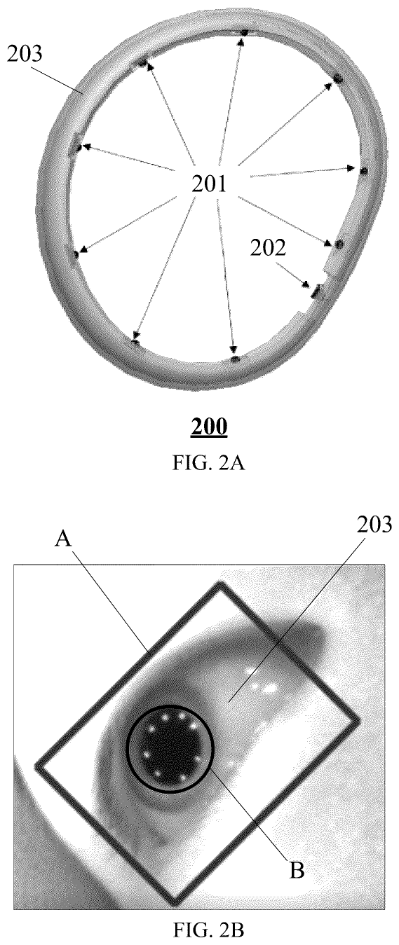

FIG. 2A illustrates a schematic structural diagram of a module for tracking an eyeball 200 provided by related technologies. A plurality of infrared LEDs 201 and an infrared camera 202 are arranged at equal intervals on a fixing member 203. Optionally, a red camera is placed in the lower left corner. Here, the lower left corner can be understood as a location below an inner eye corner of the user when the user wears the module for tracking an eyeball.

FIG. 2B illustrates a schematic diagram of a tracking image obtained by the module for tracking an eyeball provided by the related technologies. It can be understood that the tracking image in FIG. 2B is obtained using the module for tracking an eyeball shown in FIG. 2A. Specifically, the tracking image includes an eye 203 in a square box A and light spots in circular box B. It can be seen from the area of the circular box B that the light spots of respective infrared LEDs 201 in the tracking image are arranged similarly, and the correspondence between the positions of respective light spots and the infrared LEDs cannot be determined, and thus the solution efficiency is low.

In addition, in this technology, the eye tracking accuracy is directly related to the number and recognizability of LED light spots in the tracking image reflected on the eyeball. However, due to uncertain position of the eyeball, the distance between the eyeball and the module for tracking an eyeball will also change with different groups of people and wearing attitudes, thus there may be situations where the number of LED spots that can be photographed is small, which seriously reduces the solving accuracy and success rate, and even the eye posture cannot be solved properly.

In view of this, the embodiments of the present disclosure provide a module for tracking an eyeball and an extended reality device. By adding a first light source and forming a first light spot on the eye, the position of a luminescent light source corresponding to at least part of the light spots in the light spot image are determined based on the first spot, and thus light spot confusion is effectively prevented, thereby improving algorithm recognition and solving efficiency, and achieving beneficial effects of improving eye tracking accuracy, increasing the available range of eye tracking, simplifying the algorithm, and reducing system power consumption.

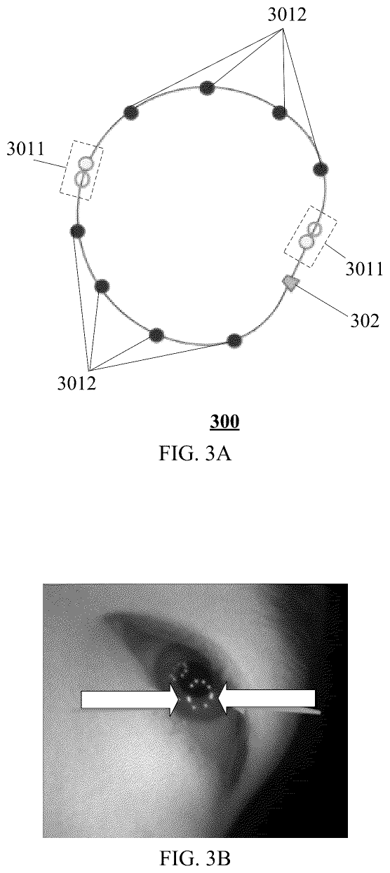

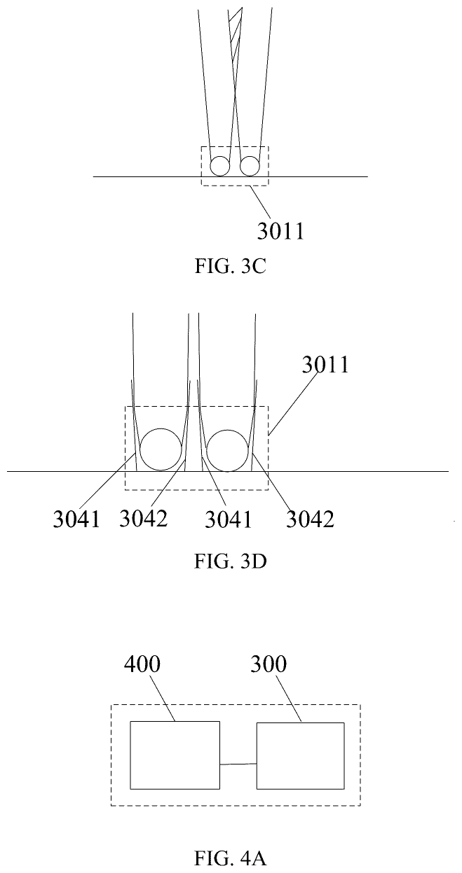

FIG. 3A illustrates a schematic structural diagram of a module for tracking an eyeball provided by an exemplary embodiment of the present disclosure.

The module for tracking an eyeball 300 includes luminescent light sources 3011, 3012, an image acquiring unit 302 and an analyzing unit (not shown in the drawings), details thereof are as follows:

The luminescent light sources 3011 and 3012 are configured to form light spots on an eye of a user. It can be understood that the luminescent light sources may be infrared LEDs, and the infrared LEDs may emit infrared light that is not perceived by human eyes, thereby not interfering with the user's perception of ambient light. At the same time, infrared light can be detected and recorded by the image acquiring unit, thereby realizing solving of eyeball pose. Herein, ambient light may be real ambient light or virtual ambient light, which is not limited in the present disclosure.

In some embodiments, luminescent light sources may be positioned around periphery of the eyeball of the user. It should be noted that the periphery of the eyeball herein includes not only position of the edge of the eyeball, but also an area within a certain range from the edge of the eyeball. For example, if the module for tracking an eyeball is disposed on a pair of VR glasses, the luminescent light sources can be arranged on a frame of the VR glasses. Optionally, the luminescent light sources are transparent infrared LEDs, and the luminescent light sources may also be arranged on the lenses of the VR glasses, which is not specifically limited in this disclosure.

It can be understood that the connecting wires among the plurality of luminescent light sources may be circular, elliptical or similar to an elliptical shape. That is to say, the exemplary embodiments of the present disclosure do not limit the distance between each luminescent light source and the pupil. Those skilled in the art can make reasonable designs based on factors such as the shape of the fixing member.

The luminescent light sources include a first light source 3011. The first light source 3011 may form a first light spot on the eye of the user, and a position of a luminescent light source corresponding to at least part of the light spots in the light spot image acquired by the image acquiring unit 302 can be determined based on the first light spot. Herein, the first light source 3011 is a position mark light source, and the first light spot formed by the first light source is a position mark light spot. Based on the first light spot, the analyzing unit may accurately locate the position of the luminescent light source which forms at least part of the light spots in the light spot image, thereby greatly improving the accuracy and recognition rate of the solution algorithm.

It should be noted that at least part of the light spots may be part of the light spots in the light spot image (for example, 1, 2, etc.), or they can be all light spots, which are not specifically limited here. Those skilled in the art may understand that after the luminescent light source corresponding to the part of the light spots are determined, positions of luminescent light sources corresponding to other light spots can be calculated using algorithms, which will not be described again here.

That is to say, by adding the first light source, the light spot of the first light source can be used to locate luminescent light sources corresponding to respective light spots in the light spot image, thereby reducing the amount of repeated algorithm calculations, simplifying the algorithm, and reducing overall power consumption.

It should be noted that the first light spot has features that can be quickly recognized by the analyzing unit, including but not limited to flicker frequency, shape, etc.

The image acquiring unit 302 is configured to acquire a tracking image of a target area. Herein, the target area at least includes an eye of the user, that is, the target area may be larger than the eye of the user to ensure that the acquired tracking image includes am eye image.

It can be understood that those skilled in the art may select an appropriate image acquiring unit based on the features of the luminescent light sources. For example, if the luminescent light sources are infrared LEDs, the image acquiring unit 302 may acquire an image of infrared light. Optionally, the image acquiring unit 302 includes an infrared camera. The number of infrared cameras may be 1 (as shown in FIG. 3A), 2, or other numbers, which is not limited in this disclosure.

The analyzing unit is configured to determine a pose of the eyeball based on an eye image and a light spot image in the tracking image. Those skilled in the art may understand that the pose of the eyeball includes information such as a direction and an attitude of the eyeball. The analyzing unit may perform functions such as image recognition and eyeball pose solving.

In some embodiments, the analyzing unit is connected to the image acquiring unit to obtain the tracking image. The analyzing unit may be a local processor or a cloud processor, which is not limited in the present disclosure. Therefore, the connection between the analyzing unit and the image acquiring unit may be wired connection or wireless connection.

The first light source 3011 is mainly configured to determine a position of a light source corresponding to at least part of the light spots, and the number of the first light sources is limited. If the luminescent light source only includes a first light source 3011, it will be difficult to position the eyeball with high precision. If the number of first light sources 3011 is increased, in order to realize the positioning function, there must be differences between the plurality of first light sources 3011, which will inevitably lead to an increase in difficulty in designing and processing the first light sources 3011, and the difficulty for the analyzing unit to identify the first light sources 3011 will also be increased. It should be noted that the light spot of the first light source may be used to determine the position of the eyeball.

Thus, in addition to the first light source 3011, the luminescent light sources also include a second light source 3012. Herein, the light spot of the second light source 3012 is mainly used to determine the pose of the eyeball, and the light spot thereof cannot be used for marking positions. The position of the second light source is estimated using the light spot of the first light source.

The first light source 3011 and the second light source 3012 form different light spots. The analyzing unit may accurately determine which light spot in the light spot image is the light spot of the first light source 3011 and which light spot is the light spot of the second light source 3012.

Different light spots may have different shapes. For example, the shape of the light spot of the first light source 3011 may be triangular, and the shape of the light spot of the second light source 3012 may be circular. It should be noted that when the light spots are distinguished based on their shapes, the greater the shape difference between the light spot of the first light source and the light spot of the second light source, the more advantageous it is for the analyzing unit to perform recognition and differentiation.

It should be understood that the different shapes of the first light source 3011 and the second light source 3012 can be realized by using different shapes of the light-emitting elements, which are not specifically limited here.

Different light spots have different flashing frequencies. For example, the flashing frequency of the light spot of the first light source 3011 is 150 Hz, and the flashing frequency of the light spot of the second light source 3012 is 100 Hz.

It should be noted that when different flicker frequencies are used to distinguish the light spots of the first light source 3011 and the second light source 3012, the sampling frequency of the image acquiring unit 302 is greater than the flicker frequencies of the light spots to ensure that the image acquiring unit 302 can accurately obtain the spot image of the first light source 3011 and the second light source 3012.

Optionally, the sampling frequency is 2 times or more than 2 times of a higher flicker frequency in the two flicker frequencies of the light spots of the first light source and the second light source, such as 3 times or 4 times, and there is no specific limit on this. Of course, if one of the two is always on, the sampling frequency is 2 times or more than 2 times of the other.

In some embodiments, different numbers of light-emitting elements are provided for the first light source 3011 and the second light source 3012, and different light spot shapes are formed with difference in the numbers of light-emitting elements. For example, each of the first light source 3011 and the second light source 3012 includes at least one light-emitting element, and the first light source 3011 and the second light source 3012 have different numbers of light-emitting elements.

In this way, the difference in the shapes of the light spots lies only in that the numbers of sub-light spots contained in respective light spots are different, thus it beneficial for the analyzing unit to perform image recognition and is beneficial in reducing the difficulty of image recognition. In addition, only the same light-emitting elements are to be purchased during the preparation process to meet product needs, which facilitates the production management, and improves production and management efficiency.

Optionally, the first light source 3011 includes at least two light-emitting elements disposed side by side, such as two, three or more. In addition, the number of the second light sources 3012 only needs to be different from that of the first light sources 3011.

For example, as shown in FIG. 3A, the first light source 3011 includes two light-emitting elements. The second light source 3012 include one light-emitting element.

Optionally, the image acquiring unit 302 of the module for tracking an eyeball includes one infrared camera. The number of first light sources 3011 is two, and each first light source 3011 includes two light-emitting elements, such as two infrared LEDs. The number of the second light sources 3012 is six, and each second light source 3012 includes one light-emitting element, such as one infrared LED.

FIG. 3B illustrates a schematic diagram of a tracking image obtained by another module for tracking an eyeball provided by an exemplary embodiment of the present disclosure. It can be understood that the tracking image in FIG. 3B is obtained by the module for tracking an eyeball shown in FIG. 3A. The position pointed by the arrow in FIG. 3B corresponds to the light spot of the first light source. It can be seen from the drawing that the shape of the first light spot is a shape of two bright spots, and the shape of the second light spot is a shape of one bright spot.

FIG. 3C illustrates a schematic structural diagram of a first light source 3011 provided by an exemplary embodiment of the present disclosure. The light emitted by the light-emitting elements arranged side by side in the first light source may overlap, resulting in a difference between the shape of the light spot and a predetermined shape, which increases the difficulty of calculation, and even makes it impossible to be effectively distinguished from the light spot formed by the second light source 3012.

Therefore, the present disclosure exemplarily provides a first light source structure that is easier to perform recognition and has higher stability. Specifically, a light modulating unit is added to the module for tracking an eyeball, and the light modulating unit is disposed in at least part of periphery areas of respective light-emitting elements in the first light source 3011, and is configured to modulate light of the light-emitting elements so that the light of a light-emitting element does not overlap with light of adjacent light-emitting elements. Though the light modulating unit, the integrity of the light spot is ensured, thereby reducing the solving difficulty and improving the solving efficiency.

FIG. 3D illustrates a schematic structural diagram of another first light source 3011 provided by an exemplary embodiment of the present disclosure. Optionally, the light modulating unit includes first reflective surfaces 3041 and second reflective surfaces 3042 disposed oppositely; wherein the first reflective surfaces 3041 and the second reflective surfaces 3042 are arranged along arrangement directions of respective light-emitting elements. The first reflective surfaces 3041 and the second reflective surfaces 3042 are arranged respectively along arrangement directions of respective light-emitting elements; and the first reflective surfaces and the second reflective surfaces are respectively perpendicular to the arrangement directions. Using the first reflective surfaces 3041 and the second reflective surfaces 3042 to adjust the light paths of respective light-emitting elements in the first light source 3011 can ensure that the sub-light spots formed by light-emitting elements in the light spot do not overlap with each other, thereby increasing the overall visibility of the light spot.

As an alternative implementation, a first reflective surfaces 3041 and a second reflective surfaces 3042 can be integrated as a whole component to wrap the outer periphery of a light-emitting element.

Optionally, the light modulating unit may also be a light-absorbing layer. For example, the outer periphery of each light-emitting element in the first light source 3011 uses a light-absorbing layer to absorb light from sides of the light-emitting element to avoid light overlapping between adjacent light-emitting elements.

In some embodiments, the number of first light sources may be one or more than one, such as two. When the number of first light sources is more than one, the plurality of first light sources are arranged symmetrically. Herein, the symmetrical arrangement may be an axially symmetrical arrangement or a centrally symmetrical arrangement, which is not specifically limited herein.

This arrangement helps ensure that the first light source covers the entire eye area and facilitates precise positioning of the second light source. When the tracking image does not include all light spots, a luminescent light source corresponding to a light spot included in the light spot tracking image of the first light source may also be used for positioning, so as to realize the pose solving of the eyeball, improve the success rate of eye tracking solving, and improve crowd and scene coverage.

In some embodiments, the light spots of the plurality of first light sources are the same or different. Herein, if the light spots of the first light sources are different, the light spots may be matched with respective first light sources, and then the light spot of the second light source can be predicted to determine the position of its corresponding second light source. When the light spots of the first light sources are the same, the distinction can be made based on the relative positions of the light spots of the first light source and the positions of the light spots of the first light sources in the tracking image (for example, above the eyeball, below the eyeball).

Optionally, the image acquiring unit 302 includes one camera, and the angle between the camera and the at least one first light source 3011 relative to the center of the eye is no greater than 10°, such as 10°, 8°, or 5°. In this way, it is ensured that the image acquiring unit 302 can obtain the light spot of at least one first light source 3011. For example, if the camera is disposed in an inner eye corner area, then at least one first light source is disposed in the inner eye corner area.

In addition, those skilled in the art can design the power supply, connection wires and other components of the module for tracking an eyeball according to actual usage requirements, which is not limited in the present disclosure.

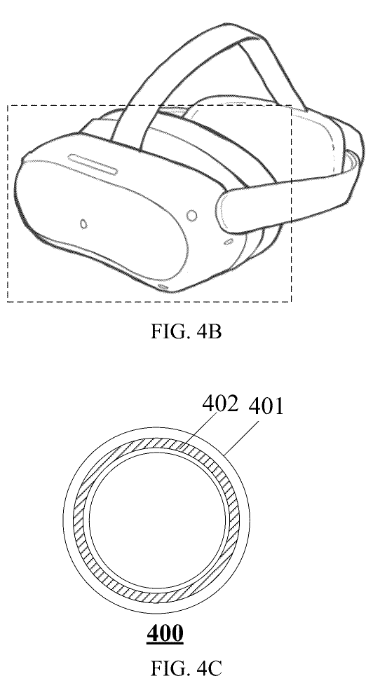

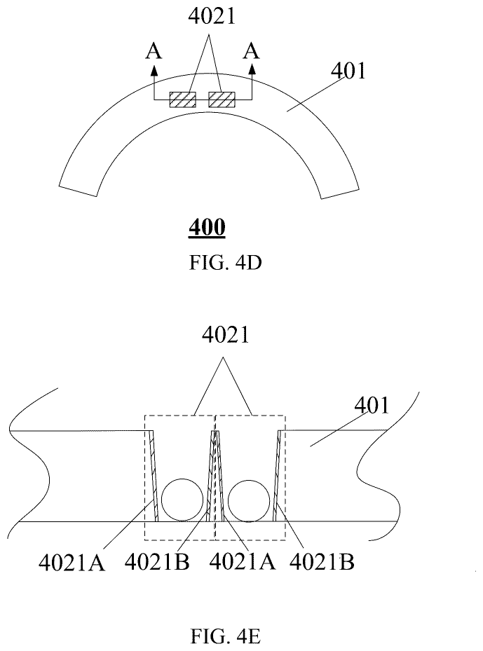

The embodiments of the present disclosure also provide an extended reality device. FIG. 4A illustrates a schematic structural diagram of an extended reality device provided by an exemplary embodiment of the present disclosure.

Specifically, the extended reality device includes a body 400 and the module for tracking an eyeball 300 disposed on the body 400 in any of the aforementioned embodiments. Here, the body 400 may include a housing or a mounting frame of the extended reality device, which is not limited in the present disclosure.

FIG. 4B illustrates a schematic structural diagram of another extended reality device provided by an exemplary embodiment of the present disclosure. The module for tracking an eyeball 300 is arranged within the dotted box in FIG. 4B.

For example, the extended reality device may be a VR all-in-one machine. The VR all-in-one machine is a device body that includes a main controller, a digital signal processor (DSP), a memory, a storage, a position sensor, a camera, a radio frequency wireless transmission circuit, an antenna and other units. Space position information is acquired through a camera, a position of the handle is obtained through a sensor and an identifier on the handle indicating a relative position to the Head-mounted Display (HMD); the radio frequency wireless transmission circuit obtains the angular velocity and gravity acceleration data of the handle, and the HMD processes the relevant data. The HMD calculates the three-dimensional position and three-dimensional angle information of the handle and HMD in space through the obtained data, and updates the image to display a handle model on the screen according to the calculated position and angle.

By using the module for tracking an eyeball 300, the user's gaze point can be determined, thereby achieving efficient gaze point rendering, which greatly improves rendering efficiency and improves clarity.

FIG. 4C illustrates a partial structural diagram of a body of an extended reality device provided by an exemplary embodiment of the present disclosure. The body is disposed in front of the eyes of the head-mounted display or VR glasses.

The body 400 includes a frame 401, and a groove 402 is disposed on the frame 401. An opening of the groove 402 faces an eye of a user, and the luminescent light sources included in the module for tracking an eye is disposed in the groove. Using the groove 402 to dispose the luminescent light sources can effectively prevent the luminescent light sources from squeezing the user's face, which improves the user's comfort.

FIG. 4D illustrates a schematic structural diagram of a body of another extended reality device provided by an exemplary embodiment of the present disclosure; FIG. 4E illustrates a schematic cross-sectional view along plane A-A in FIG. 4D.

Optionally, the groove 402 may be of a continuous annular shape, as shown in FIG. 4C, or may include a plurality of sub-grooves, as shown in FIG. 4D.

In some embodiments, the groove 402 includes at least two adjacent sub-grooves 4021, and side walls of the sub-grooves 4021 are configured to dispose a light modulating unit. It should be noted that the at least two adjacent sub-grooves 4021 are configured to dispose the first light source. Herein, the number of light-emitting elements in the first light source are used to determine the number of sub-grooves 4021 in adjacent sub-grooves; and the number of first light sources may be used to determine the number of adjacent sub-grooves 4021.

As shown in FIG. 4E, a first side wall 4021A of the groove 4021 is used as the first reflective surface, and the second side wall 4021B is used as the second reflective surface. The first reflective surface and the second reflective surface can adjust a light path of a light-emitting element installed in the sub-groove 4021 to prevent light from two adjacent light-emitting elements from overlapping and thus cannot form an effective position marking light spot.

Using the side walls of the sub-groove 4021 to dispose the light modulating unit can ensure the simplicity of the overall structure of the extended reality device while meeting the eye tracking requirements, and thereby improves the space utilization of the extended reality device.

The extended reality device of the above embodiments has the module for tracking an eyeball in any of the aforementioned embodiments, and has the beneficial effects of the corresponding method embodiments, which will not be described again here.

Those of ordinary skill in the art should understand that the above discussion of any embodiments is only illustrative, and it is not intended to imply that the scope of the present disclosure (including claims) is limited to these examples; under the idea of the present disclosure, the above embodiments or the technical features of different embodiments may be combined, and the steps can be implemented in any order. There are many other variations for different aspects of the disclosed embodiments as described above, which are not provided in detail for the sake of brevity.

Additionally, to simplify description and discussion, and in order to make the understanding of the embodiments of the present disclosure easier, well-known power/ground connections to integrated circuit (IC) chips and other components may or may not be shown in the drawings provided. Furthermore, apparatuses may be shown in block diagram form in order to make the understanding of the embodiments of the present disclosure easier. The following facts are also taken into account, that is, the details regarding the implementations of these block diagram apparatuses are highly dependent on the platform on which the disclosed embodiments are to be implemented. (i.e., these details should be within the understanding of those skilled in the art). When specific details (e.g., circuits) are set forth to describe exemplary embodiments of the present disclosure, it will be apparent to one skilled in the art that embodiments of this disclosure may be implemented with or without variations in these specific details. Accordingly, these descriptions should be considered illustrative rather than restrictive.

Although the present disclosure has been described in conjunction with specific embodiments thereof, many substitutions, modifications and variations of these embodiments will be apparent to those of ordinary skill in the art from the foregoing description. For example, the embodiments discussed above may be used in other memory architectures (e.g., dynamic RAM (DRAM)).

The embodiments are intended to cover all such substitutions, modifications and variations that fall within the broad scope of the appended claims. Therefore, any omissions, modifications, equivalent substitutions, improvements, etc. made within the spirit and principles of the embodiments of this disclosure shall be included in the protection scope of this disclosure.

I/We claim:

1. A module for tracking an eyeball, comprising:
luminescent light sources configured to form light spots on an eye of a user;
an image acquiring unit configured to acquire a tracking image of a target area; and
an analyzing unit configured to determine a pose of the eyeball based on an eye image and a light spot image in the tracking image;
wherein the luminescent light sources comprise a first light source and form a first light spot on the eye, so as to determine a position of a luminescent light source corresponding to at least part of light spots in the light spot image based on the first light spot.

2. The module for tracking an eyeball of claim 1, wherein the luminescent light sources further comprise a second light source, and the first light source and the second light source form different light spots.

3. The module for tracking an eyeball of claim 2, wherein the different light spots comprise at least one of different shapes and different flicker frequencies.

4. The module for tracking an eyeball of claim 2, wherein each of the first light source and the second light source comprises at least one light-emitting element, wherein the number of the light-emitting elements included in the first light source is different from that in the second light source.

5. The module for tracking an eyeball of claim 1, wherein the first light source comprises at least two light-emitting elements disposed side by side.

6. The module for tracking an eyeball of claim 5, further comprising:
a light modulating unit disposed in at least part of periphery areas of respective light-emitting elements in the first light source and configured to modulate light of the light-emitting elements so that the light of a light-emitting element does not overlap with light of adjacent light-emitting elements.

7. The module for tracking an eyeball of claim 6, wherein the light modulating unit comprises first reflective surfaces and second reflective surfaces disposed oppositely; wherein
the first reflective surfaces and the second reflective surfaces are arranged along arrangement directions of respective light-emitting elements, and the first reflective surfaces and the second reflective surfaces are respectively perpendicular to the arrangement directions.

8. The module for tracking an eyeball of claim 1, wherein the luminescent light source comprises at least two first light sources; wherein,
a plurality of the first light sources are arranged symmetrically; and/or
light spots of the plurality of first light sources are the same or different.

9. The module for tracking an eyeball of claim 8, wherein the image acquiring unit comprises a camera, and an angle between the camera and the at least one first light source relative to a center of the eye is not greater than 10°.

10. An extended reality device, comprising:
a body; and
a module disposed on the body and configured for tracking an eyeball, the module for tracking the eyeball comprising:
luminescent light sources configured to form light spots on an eye of a user;
an image acquiring unit configured to acquire a tracking image of a target area; and
an analyzing unit configured to determine a pose of the eyeball based on an eye image and a light spot image in the tracking image;
wherein the luminescent light sources comprise a first light source and form a first light spot on the eye, so as to determine a position of a luminescent light source corresponding to at least part of light spots in the light spot image based on the first light spot.

11. The extended reality device of claim 10, wherein the luminescent light sources further comprise a second light source, and the first light source and the second light source form different light spots.

12. The extended reality device for tracking an eyeball of claim 11, wherein the different light spots comprise at least one of different shapes and different flicker frequencies.

13. The extended reality device of claim 11, wherein each of the first light source and the second light source comprises at least one light-emitting element, wherein the number of the light-emitting elements included in the first light source is different from that in the second light source.

14. The extended reality device of claim 10, wherein the first light source comprises at least two light-emitting elements disposed side by side.

15. The extended reality device of claim 14, wherein the module for tracking the eyeball further comprises:
a light modulating unit disposed in at least part of periphery areas of respective light-emitting elements in the first light source and configured to modulate light of the light-emitting elements so that the light of a light-emitting element does not overlap with light of adjacent light-emitting elements.

16. The extended reality device of claim 15, wherein the light modulating unit comprises first reflective surfaces and second reflective surfaces disposed oppositely; wherein
the first reflective surfaces and the second reflective surfaces are arranged along arrangement directions of respective light-emitting elements, and the first reflective surfaces and the second reflective surfaces are respectively perpendicular to the arrangement directions.

17. The extended reality device of claim 10, wherein the luminescent light source comprises at least two first light sources; wherein,
a plurality of the first light sources are arranged symmetrically; and/or
light spots of the plurality of first light sources are the same or different.

18. The extended reality device of claim 17, wherein the image acquiring unit comprises a camera, and an angle between the camera and the at least one first light source relative to a center of the eye is not greater than 10°.

19. The extended reality device of claim 10, wherein the body comprises a groove, an opening of the groove faces an eye of a user, and the luminescent light sources included in the module for tracking an eye is disposed in the groove.

20. The extended reality device of claim 19, wherein the groove comprises at least two adjacent sub-grooves, and side walls of the sub-grooves are configured to dispose a light modulating unit.
